(19) United States
(12) Patent Application Publication
(10) Pub. No.: US 2026/0092067 A1
(43) Pub. Date: Apr. 2, 2026

(54) METHOD FOR PREPARING AMIDINES FROM N-(ALKYL LACTAMS)

(71) Applicant: VERSALIS S.P.A., San Donato Milanese (MI) (IT)

(72) Inventors: Nicola VECCHINI, Mantova (MN) (IT); Federico MONDINI, Mantova (MN) (IT)

(21) Appl. No.: 19/108,987

(22) PCT Filed: Sep. 4, 2023
(86) PCT No.: PCT/IB2023/058741

§ 371 Date: Mar. 5, 2025

(30) Foreign Application Priority Data
Sep. 7, 2022 (IT) 102022000018231

(51) Int. Cl. C07D 487/04

(52) U.S. Cl. CPC C07D 487/04

(57) ABSTRACT

A method for preparing amidines or derivatives thereof includes the step of subjecting N-(amino-alkyl) lactams to dehydration in the presence of a heterogeneous catalyst chosen from a Lewis acid, Lewis acids with Bronsted acid or Bronsted acid components such as cerium oxide catalysts; boron phosphates, cerium, zirconium; other zirconium salts; modified zirconium oxides; and phosphotungstic acid.

### CROSS-REFERENCE TO RELATED APPLICATIONS

This application is a 35 U.S.C. § 371 National Stage patent application of PCT/IB2023/058741, filed on 4 Sep. 2023, which claims the benefit of Italian patent application 102022000018231, filed on 7 Sep. 2022, the disclosures of which are incorporated herein by reference in their entirety.

### TECHNICAL FIELD

The present disclosure relates to a process for the preparation of amidines. More particularly, the present disclosure relates to a method for producing amidines such as, for example, 1,8-Diazabicyclo-[5.4.0]-undec-7-ene (henceforth referred to in the abbreviated form DBU), or derivatives thereof from lactams, such as ε-Caprolactam and α,β unsaturated nitriles, such as acrylonitrile.

### BACKGROUND

It is well known that DBU is a versatile molecule that lends itself to numerous applications; indeed, the chemical reactions in which it can take part are varied. A recent article by Jacques Muzart, “DBU: A Reaction Product Component′ Chemistry Select 2020, vol. 5, 11608-11620, gives a detailed summary of this, ranging from the formation of salts to addition on C-C double bonds and much more. For these aspects DBU is used in the catalysis of polyurethanes, in the pharmaceutical industry, in ionic liquids and in general in organic synthesis. Further details on the application of DBU are also described in “1,8-Diazabicyclo[S.4.0]undec-7-ene (DBU): A Versatile reagent in Organic Synthesis” Bhaskara Nand et al. Current Organic Chemistry, 2015, 19, 790-812. In the prior art, the industrial production of DBU takes place mainly through three reaction steps. In the first step ε-caprolactam is reacted with acrylonitrile to obtain N-(2-cyanoethyl)-ε-caprolactam. In the second step, N-(2-cyanoethyl)-ε-caprolactam is hydrogenated to the corresponding amine in the presence of anhydrous ammonia and Nickel Raney catalyst. In the third step, N-(3-aminopropyl)-ε-caprolactam is dehydrated by acid catalysis to produce DBU. The industrially most complex step in the synthesis is hydrogenation step in the presence of ammonia. The catalyst normally used is Nickel-Raney which in its activated form is pyrophoric. Anhydrous ammonia is also a toxic gas and requires specific precautions and authorisations for its storage, use and transport.

Below are some relevant documents of the prior art.

Patent DE1545855 describes a process for obtaining amidines (limited to the third step of the industrial process described above) with the following structure:

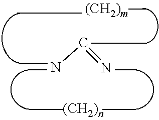

where m is an integer from 3 to 7, and n is an integer from 2 to 4, starting from N-(aminoalkyl) lactams of formula:

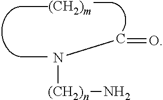

The process takes place through the dehydration of aminolactam catalysed by mineral or sulfonic acids (e.g. p-toluenesulfonic acid) in the presence of a solvent e.g. Xylene. The reaction mixture is heated to boiling point, the dehydration water formed is condensed together with the solvent and then separated; the solvent is refluxed into the reaction flask. The patent does not describe the steps prior to dehydration, but makes reference to the prior art.

Patent application EP0347757 A2 describes a method for the synthesis of cyanoalkyl lactams (the first step in the industrial process described above) through the reaction of a lactam and an α,β unsaturated nitrile using DBU itself as a basic catalyst; DBU can also be used as a solvent. The document does not mention the other reaction steps (second and third), but merely refers to the catalytic hydrogenation of cyanoalkyl lactam as in the prior art; indeed, in example 2, hydrogenation in the presence of Ni Raney and ammonia is described. In the patent application, the use of DBU as a catalyst is justified as a substitute for KOH used in the first step of the industrial process, since, in order to be able to pass from the first step to the second, the base must first be neutralised, which is not necessary if DBU is used (example 3).

Patent CN101279973 B describes a method for the preparation of 1,8-Diazabicyclo-[5.4.0]-undec-7-ene, starting from ε-caprolactam and acrylonitrile, in the presence of tert-butyl or tert-amyl alcohol, as a solvent, and NaOH as a catalyst. The reaction product of this first step undergoes hydrogenation in the presence of anhydrous ammonia and Ni Raney as a catalyst. After hydrogenation, the mixture is neutralised with sulfuric acid, the solvent recovered and the reaction product is subjected to dehydration, with removal of water, as described in German patent DE1545855.

Patent publication CN109796458 A describes a method for the preparation of 1,8-Diazabicyclo-[5.4.0]-undec-7-ene, again starting from ε-caprolactam and acrylonitrile. This time, the document no longer describes the hydrogenation step in the presence of ammonia, but introduces an alternative method using hydroquinone, anhydrous gaseous hydrochloric acid, dichloromethane, sodium perborate and ethylenediaminetetraacetic acid (EDTA). The process is considerably more complex than the others described, and while ammonia and Ni-Raney are eliminated, a highly aggressive agent (anhydrous HCl) is introduced as well as numerous chemical substances.

In patent publication JP2003286257, the first and third steps are carried out in the same way as described above (reaction of caprolactam with acrylonitrile by basic catalysis using KOH; dehydration by acid catalysis). The second step is conducted in the absence of ammonia and using Cobalt Raney as a catalyst. The result is 86% by weight of reduced product (primary amine of interest).

Patent EP0913388 B1 describes a method for obtaining amines by hydrogenation of nitriles without the use of ammonia. The novelty lies in the treatment to which the catalyst is subjected. The catalyst (Cobalt Raney or sponge catalyst) is treated with an aqueous lithium hydroxide solution or, alternatively, the reaction is carried out in the presence of this solution. Through this treatment, the catalyst must incorporate 0.1 to 100 mmol of lithium hydroxide per gram.

Patent EP0662476 B1 describes the synthesis of bicyclic amidines by acid-catalysed reaction of lactones with diamines. The process is carried out in a single reaction step and is followed by purification. The patent also claims the use of these amidines as catalysts for polyurethanes. The synthesis of DBU is described in Example 6 and shows a very low product yield of 21%.

Patent publication CN1262274 A describes a method for the preparation of 1,8-diazabicyclo-[5.4.0]-undec-7-ene from ε-caprolactam and acrylonitrile, the special feature being the use of a mixture of inorganic and organic bases as catalysts in the first reaction step (KOH and DBU). The cyano-derivative obtained is subjected to purification before reduction. The second hydrogenation step is carried out in the presence of activated Ni (the catalytic form is not defined) as a catalyst but it is not mentioned whether in the presence or absence of ammonia. Dehydration is always carried out under acidic conditions, using p-toluenesulfonic acid, and in the absence of a solvent; the reaction is carried out over a fairly long period, i.e. between 35 and 40 hours, obtaining a yield for this step of 74.61%.

According to the prior art, the final cyclization step to give the bicyclic amidine is conducted with an acid catalyst, specifically p-toluenesulfonic acid, which is dissolved in the reaction environment and which requires the use of a high-boiling solvent to conduct the reaction. This results in greater complexity of the production plant and process—also in relation to the need to separate the catalyst from the reaction mixture—and a consequent increase in costs.

### SUMMARY

The aim of the present disclosure is therefore to provide an innovative process for the synthesis of amidines that enables simplification and reduction of plant, process and maintenance costs.

This is achieved through the use of appropriate heterogeneous catalysis in the dehydration/cyclization reaction, resulting in the elimination of both the solvent reflux and the neutralisation phase of the mixture (necessary if homogeneous catalysis is used, e.g. with p-toluenesulphonic acid as a catalyst).

In particular, the present disclosure prepares 1,8-Diazabicyclo-[5.4.0]-undec-7-ene (DBU)—usable in the applications described above—from the corresponding N-(amino-alkyl) lactam, by using heterogeneous catalysis and consequent elimination of solvent reflux and neutralisation.

The Applicant therefore set out to find a process for the production of amidines from N-(amino-alkyl) lactams.

The Applicant has now found a method for the preparation of amidines from N-(amino-alkyl) lactams, comprising dehydration/cyclization of the amine compound to obtain the amidine, which can then undergo a final stage of separation and purification to obtain the product in a form suitable for industrial use. This method can be conducted in batch or continuous mode; continuous mode is preferred.

N-(amino-alkyl) lactams can be prepared using one of the processes described in the state of the art, such as those described above, or, more preferably, according to the method described in Italian patent application number 1020210005321 entitled “METHOD FOR PREPARING AMIDINES” by the same Applicant, filed on 8 Mar. 2021 and incorporated herein in its entirety for reference purposes.

The reduction of the nitrile compound, which is the most critical step in the synthesis of N-(aminoalkyl)lactams, is a reaction already known and reported in literature and widely used in organic synthesis (see, for example, Peter Vollhardt, Organic Chemistry p. 825-826 1st Edition). In the patents listed above, it is conducted using Raney catalysts (Ni or Co) or otherwise in sponge form, and in the presence of anhydrous ammonia.

Suitable reduction catalysts described in the above-mentioned Italian patent application number 1020210005321 are commercial or synthetic hydrogenation systems based on one or more metals from groups 8, 9 and 10 of the periodic table, such as Iron, Cobalt, Nickel, or noble metals such as Ruthenium, Rhodium, Palladium, Osmium, Iridium or Platinum. Cobalt, Nickel, Palladium and Platinum are preferred. Cobalt and Nickel are particularly preferred. Such catalysts can be used in the dispersed, colloidal or supported/bonded phase, preferably in supported/bonded form on an inorganic phase with a high surface area, even more preferably in a supported/bonded phase on silica, alumina or silica-alumina.

Surprisingly, the Applicant has found that it is possible to conduct the synthesis of amidines with series reactions, eliminating the solvent prior to the cyclization/dehydration step and carrying out a single final purification step without the process presenting any critical issues, or requiring separation steps of the intermediates of the desired product from the other reaction products, to guarantee an acceptable final purity of the desired product and a high yield and conversion into the desired product in each of the intermediate steps. This simplifies the number of devices to be used and considerably reduces the complexity of the overall process. Optionally, the use of intermediate purification steps can be considered if high-purity semi-finished products and/or chemical intermediates are required.

These and other purposes are surprisingly achieved by the preparation process in accordance with the present disclosure.

### DETAILED DESCRIPTION OF THE DISCLOSURE

The present disclosure therefore provides a process for the preparation of amidines or derivatives thereof of formula (V)

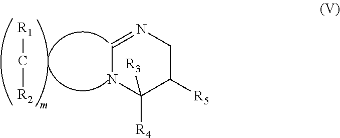

from an N-(amino-alkyl) lactam having the following formula (IV)

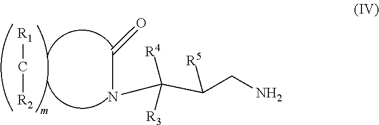

wherein.
R1 is H or an aliphatic hydrocarbon group, optionally substituted, having from 1 to 5, preferably from 1 to 2, carbon atoms, and is more preferably H;
R2 is H or an aliphatic hydrocarbon group, optionally substituted, having from 1 to 5, preferably from 1 to 2, carbon atoms, and is more preferably H;
R3 is H or an aliphatic hydrocarbon group, optionally substituted, having from 1 to 5, preferably from 1 to 2, carbon atoms, and is more preferably H;
R4 is H or an aliphatic hydrocarbon group, optionally substituted, having from 1 to 5, preferably from 1 to 2, carbon atoms, and is more preferably H;
R5 is H or an aliphatic hydrocarbon group, optionally substituted, having from 1 to 5, preferably from 1 to 2, carbon atoms, and is more preferably H;
m is an integer from 3 to 7, more preferably from 3 to 6,
   wherein even more preferably, (V) is 1,8-Diazabicyclo [5.4.0]undec-7-ene, (DBU),
   said process comprising the following step:
   subjecting said amine of formula (IV) to dehydration, with a solid catalyst based on compounds containing a metal or semi-metal chosen from boron, cerium, tungsten, zirconium or possibly rare earths other than cerium, such as, for example, catalysts based on, or comprising, at least one compound chosen from oxides of boron, cerium, tungsten; phosphates or other salts of boron, cerium, tungsten; phosphates of rare earths other than cerium; zirconium salts or modified zirconium oxides, phosphotungstic acid, obtaining the corresponding amidine of formula (V).

The amidine of formula (V), synthesised as described above in accordance with the present disclosure, may be subjected to subsequent purification by methods known to a person skilled in the art.

In accordance with the present disclosure, the term “amidine” means a compound derivable from an amide by substitution of the oxygen of the carbonyl group ═CO by an imide group ═N—. Preferably, the present disclosure also considers cyclic amidines as defined in formula (V).

In accordance with the present disclosure, the term “amidine derivative” means any compound obtainable from an amidine by reaction with a carboxylic acid, an epoxyketone, chloroformates or carbonic acid diesters.

In accordance with the present disclosure, the singular indefinite article, one, is understood to include also the meaning of at least one, unless otherwise specified.

In this document, unless otherwise indicated, percentages are to be understood as mass percentages.

In accordance with the present disclosure, the term “based on” is intended to identify not only catalysts that “consist of” elements indicated as catalytically active, but also catalysts that “comprise” them.

The amino-derivative of the lactam of formula (IV) according to the present disclosure is subjected to dehydration, by heterogeneous catalysis, to give the corresponding amidine, in the preferred case DBU (1,8-Diazabicyclo [5.4.0]undec-7-ene), as described below.

The amino-derivative of the lactam of formula (IV) which undergoes the dehydration process according to the present disclosure may be pure or may be in a mixture with the solvent and/or the products of the hydrogenation reaction by which the amino-alkyl lactam of formula (IV) is obtained from its corresponding cyano-alkyl lactam.

The reaction mixture from the hydrogenation stage is preferably subjected to solvent recovery by evaporation, followed by dehydration.

In another preferred form, the reaction mixture from the hydrogenation step undergoes dehydration and then solvent recovery by separating it from the water formed by the reaction. Alternatively, although in a less preferable embodiment, the amino derivative of the lactam of formula (IV) can be reacted in purified form.

The dehydration is carried out under warm/hot conditions, preferably between 9° and 270° C., more preferably between 13° and 230° C., even more preferably between 150 and 200° C., continuously removing the water produced during the dehydration process which operates the cyclization.

The catalyst in dehydration is always necessary and can be chosen from heterogeneous acid catalysts chosen from Lewis acids, Lewis acids with Bronsted acid components, or Bronsted acids such as catalysts based on, or comprising, at least one compound chosen from
cerium oxide (ceria) or its salts, preferably phosphates such as for example cerium phosphate, cerium pyrophosphate;
phosphates of rare earths other than cerium, e.g. lanthanum;
boron phosphate;
phosphotungstic acid;
zirconium salts, preferably phosphates, or modified zirconium oxides e.g. zirconium phosphate, sulfated zirconium oxide; and
combinations thereof.

In an embodiment, the catalyst is based on, or comprises, one or more compounds chosen from among
cerium oxide (ceria) and/or its salts, preferably phosphates;
boron phosphate;
phosphotungstic acid;
zirconium salts, preferably phosphates, and/or modified zirconium oxides, preferably sulfated zirconium oxide.

Boron phosphate, cerium oxide, sulfated zirconium oxide, zirconium salts or combinations thereof are preferred.

In an embodiment, zirconium sulfate can be used as modified zirconium oxides.

Catalysts based on, or comprising, at least one compound chosen from boron phosphate, sulfated zirconium oxide and zirconium salts, or combinations thereof, are most preferred.

Said catalysts based on, or comprising, one or more of the aforementioned catalytically active compounds may also comprise further components in that they may be optionally supported on inert “carriers” and/or bonded with said carriers (for forming the catalyst) such as, for example, pumice, graphite or silica, although other carriers known in the art may be employed without departing from the scope of the present disclosure.

The catalyst based on, or comprising, boron phosphate can be prepared according to techniques known to a person skilled in the art from the reaction of boric acid with phosphoric acid, as for example described in the examples in the present application.

The catalyst based on, or comprising, sulfated zirconium oxide (“Zirconium oxide, catalyst support, sulfated”; “zirconium oxide sulfated”; “sulfated zirconia”) may be a commercial product or may be prepared according to known techniques, e.g. as reported on page 198 in the article by F. Cavani et al. “The control of selectivity in gas-phase glycerol dehydration to acrolein catalysed by sulfated zirconia”, Applied Catalysis B: Environmental 100 (2010) 197-204.

The catalyst based on, or comprising, cerium oxide can be prepared by techniques known in the art, for example as described in the examples in the present application or as described in U.S. Ser. No. 10/894,750B2.

At the end of the reaction the main dehydration product is the amidine of interest of formula (V).

If required by end-users, the amidine can be purified by one of the methods already known in the state of the art, e.g. by distillation to a purity which varies from 95 to 98% by weight.

Preferably, in the process of the present disclosure, no intermediate purification of the reaction mixture obtained from hydrogenation is carried out, but only the evaporation of any solvent for the recovery and use thereof.

The Applicant has therefore surprisingly identified the possibility of operating solvent-free dehydration on solid acid catalyst without refluxing the solvent, in order to facilitate water removal, with further simplification of the process and reduction of costs compared to the use of p-toluenesulfonic acid. However, the use of a solvent typically chosen from those used in known hydrogenation reactions to provide the intermediate of formula (IV), e.g. a xylene, is not excluded.

In the present process, the reaction step and the final purification step can be conducted continuously.

In a particularly preferred embodiment of the present disclosure, the Applicant has found a novel and original process for producing amidines from lactams.

All conversion, selectivity and yield values mentioned refer to those determined by ¹H and ¹³C NMR and GC-MS analysis of the reaction mixtures as described in the examples.

The reagent stream from the hydrogenation step can be sent to a solvent recovery system. The preferred set-up is one based on an evaporator for solvent recovery. The reaction mixture with traces of solvent comes out of the bottom of the evaporator. The steam from the evaporator is fed to the degasser, which contains perforated plates to facilitate both the separation and contact of the two phases, liquid and steam. The vapour phase leaving the degasser is partially condensed in a reflux condenser, which operates at a temperature of 20-250° C., preferably 40-150° C., even more preferably at 60-130° C.; optionally; further condensation can optionally be carried out to recover any by-products formed during the previous reactions.

The vapours leaving the reflux condenser are condensed in another condenser at a temperature comprised between 2-50° C. preferably 10-30° C., more preferably 20° C.

The liquid that collects at the outlet of the last condenser is a mixture of solvent and water. The solvent, after water separation, can be recycled, while the mixture leaving the bottom of the evaporator can be sent to the dehydration step.

Dehydration takes place continuously in a reactor, called a dehydrator, preferably of the tubular type, equipped with a heating system and a condensing system consisting of a post-condenser which condenses most of the water produced and sends the condensates to a phase separator. In the phase separator, any traces of organic matter are separated and reintroduced into the dehydrator, while the water can be partly recycled to a hydrogenation section that can be arranged upstream and the excess sent for treatment. In a preferred form, the mixture is continuously fed laterally into the reactor, while steam comes out of the reactor head and the reaction product from the bottom. This reactor may optionally contain fillings (packings) in the upper part, such as rings, plates, septa, so that only water vapour can escape.

In another embodiment, the reaction mixture can also be continuously fed from the bottom and the reaction product taken from the side of the reactor while water vapour escapes from the reactor head.

The reaction is conducted in the presence of a heterogeneous acidic catalyst, as defined above, with a WHSV (Weight Hourly Space Velocity, relative to the entire reagent mixture) comprised between 1 and 50 h⁻¹, preferably between 3 and 10 h⁻¹.

Dehydration is carried out under warm/hot conditions, preferably between 9° and 270° C., more preferably between 13° and 230° C., even more preferably between 15° and 200° C.

The pressure at which the reaction is conducted is between 0.08 and 5 BarA, preferably between 0.5 and 3 BarA, more preferably between 1 and 2 BarA.

A stream consisting of the dehydration products, any unreacted solvent and amine, and the by-products of the previous steps exits from the bottom of the reactor; where the compound of formula (IV) is N-(3-aminopropyl)-ε-caprolactam, the main product is typically DBU (1,8-Diazabicyclo [5.4.0]undec-7-ene).

Said product stream is then sent to a distillation section for purification of the compound (V), such as DBU. After distillation, the purity of said compound is typically comprised between 95 and 98%.

The purity of said compound is determined by gas chromatography analysis (GC-MS).

Optionally, said compound after distillation can be subjected to further purification such as liquid-liquid extractions. Such operations can be carried out using techniques known to a person skilled in the art.

As mentioned above, the aforementioned N-(amino-alkyl) lactam compound of formula (IV) to be subjected to the dehydration process according to the present disclosure can be prepared using one of the processes described in the state of the art, such as those previously described in the background of the present disclosure, or, more preferably, according to the method described in Italian patent application number 1020210005321 entitled “METHOD FOR PREPARING AMIDINES” by the same Applicant, filed on 8 Mar. 2021 and incorporated herein in its entirety, in particular with reference to the reduction step (B).

In the above-mentioned Italian patent application, N-(amino-alkyl) lactam of formula (IV) is obtained by subjecting a nitrile lactam compound of formula (III)

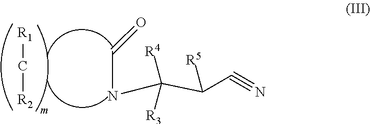

where R1, R2, R3, R4 and R5 have the meanings defined above for formula (IV), (V),
   to reduction of the nitrile group by reaction with hydrogen in the presence of a catalyst based on metals of groups 8, 9 and 10 of the periodic table, e.g. iron, cobalt, nickel, or noble metals such as ruthenium, rhodium, palladium, osmium, iridium or platinum, where said catalyst is not a Raney or sponge-type catalyst, to obtain the corresponding amine of formula (IV).

Cobalt, Nickel, Palladium and Platinum are preferred. Cobalt and Nickel are particularly preferred.

Such reduction catalysts can be used in the dispersed, colloidal or supported/bonded phase, preferably in supported/bonded form on an inorganic phase with a high surface area, even more preferably in a supported/bonded phase on silica, alumina or silica-alumina, cobalt on alumina support being particularly preferred.

In a preferred embodiment, the reduction catalysts are Co- and Ni-based catalysts, preferably supported/bonded on a Lewis acid or a Lewis acid having Bronsted acid components, more preferably Al₂O₃ or SiO₂, wherein said catalyst is not a Raney or sponge-type catalyst.

The reduction step, hereinafter also referred to as step (B), is conducted using said reduction catalyst, with H₂ and in the absence of ammonia, in the presence of water in a molar ratio of H₂O/(III) comprised between 0.01 and 1 or in a weight ratio between 0.1 and 11% with respect to the reactant mixture.

The reaction temperature of step (B) is comprised between 3° and 250° C., preferably between 5° and 200° C., and the pressure is comprised between 4 and 5150 barA, preferably between 11 and 100 barA, even more preferably between 20 barA and 60 barA.

The reduction reaction may be conducted in batch (in a reactor equipped with a stirrer, heating jacket and inlets for gases and liquid streams) for a reaction time of 0.1 to 12.0 h, preferably comprised between 0.8 and 7.0 h, more preferably from 1.5 to 5 h; or, it may be conducted continuously, e.g. in a single- or multi-stage tubular reactor or in a stirred reactor such as a CSTR. Continuous mode is preferred for productivity issues, particularly on an industrial scale.

The reduction reaction of step (B) can be conducted in the presence of an organic solvent. In one embodiment, said organic solvent is preferably chosen from methanol or ethanol, isopropyl or tert-butyl alcohol, MTBE, THF, more preferably THF. In another embodiment, the organic solvent is an aromatic solvent such as benzene, toluene, xylenes, ethylbenzene, most preferably xylene (o, m, p or mixture of isomers) and ethylbenzene.

The compound of formula (III) to be used in the aforesaid step (B) is preferably obtained by reacting a lactam having the following formula (I)

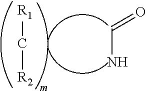

and an unsaturated α,β nitrile having the following formula (II)

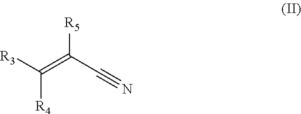

under addition reaction conditions, by one of the methods known to those skilled in the art in the presence of a suitable basic catalyst, preferably KOH, NaOH and LiOH, obtaining a compound of formula (III) as defined above. This addition reaction step is hereinafter also referred to as step (A).

Preferably in step (A), the lactam of formula (I) is ε-caprolactam, and the a (3-unsaturated nitrile of formula (II) is acrylonitrile.

The present disclosure further provides a process for the preparation of amidines or their derivatives of formula (V) as defined above, comprising the following steps:
synthesis of nitrile lactams of formula (III) as defined above by reaction between a lactam of formula (I) and an α-β-unsaturated nitrile of formula (II) as defined above according to the procedure of step (A) described above;
synthesis of N-(amino-alkyl) lactams of formula (IV) as defined above, subjecting said nitrile lactams of formula (III) as defined above to reduction with H₂ according to the process of step (B) described above;
synthesis of amidines of formula (V) as defined above, by subjecting said N-(amino-alkyl) lactams of formula (IV) as defined above to dehydration according to the dehydration process described above in accordance with the present disclosure.

Some illustrative but not limiting examples of the present disclosure now follow.

### EXAMPLES

In the following examples, the following abbreviations and materials are used, unless otherwise indicated:
AN: acrylonitrile (CAS 107-13-1, purity≥99%, Sigma-Aldrich)
CPLT: ε-caprolactam (CAS 105-60-2, purity 99%, Sigma-Aldrich)
NaOH: sodium hydroxide (CAS 1310-73-2, purity≥98%, Sigma-Aldrich)
Xylene: mixture of xylene isomers (CAS 1330-20-7, purity≥98.5%, Sigma-Aldrich)
CTZ1: commercial catalyst HTC CO 2000 RP 1.2 mm (Co≈15% supported on alumina) Johnson-Matthey (data from U.S. Pat. No. 8,293,676 B2 Table 3 columns 21-22 Example J)
H₂: hydrogen (Sapio, Title 5.5)
H₂O: ultra-pure water (MilliQ millipore system)
NH₂ (in Table 1): N-(3-aminopropyl)-ε-caprolactam

### Gas-Mass Analysis

The gas-mass analysis for the determination of reagents and reaction products is carried out with a GC HP6890 chromatograph, equipped with a split/splitless injector and interfaced to an MS HP 5973 mass spectrometer acting as a detector. The chromatograph features an HP-1MS UI capillary column (100% poly dimethylsiloxane, Agilent J&W), fused silica WCOT, 30 m length, 0.25 mm ID, film thickness 0.25 μm.

The instrumental parameters are as follows:
injection volume 20 μl
Helium carrier gas at 0.8 ml/min (constant flow mode)
splitting ratio 250:1
Injector temperature 300° C.
   programmed oven temperature from 40° C. to 320° C. at 10° C./min (28 min) plus hold time of 10 min at 320° C. (total run time=38 min). In the absence of the market availability of certain pure products (the amine of ε-caprolactam), quantification was performed by the method of comparing the relative areas of the various chromatography peaks (thus accepting the approximation that they all have the same chromatography response).

However, a quantitative ¹H e ¹³C NMR analysis was also carried out on the same samples, and the results obtained were superimposable on those shown with the chromatography technique.

### NMR Analysis

Analyses on the samples provided were carried out using a Bruker Avance 400 MHz spectrometer at a temperature of 300 K by dissolving approximately 50-70 mg of sample in deuterated chloroform. The spectra were recorded with the following instrumental parameters:

| | PROBE: 5 mm PABBO BB/19F-1H/D Z-GRAD | | | |
|---|---|---|---|---|
| | H - 400 MHz | | C - 100 MHz | |
| Method | zg30 | | zgpg30 | |
| No. of scans | 64 | | 512 | |
| No. of data points | 32k | | 32k | |
| p1 (μs) | 26.00 | | 17.00 | |
| d1 (s) | 7.00 | s | 8.00 | s |
| Spectral window | 15 | ppm | 240 | ppm |
| O1P | 4.0 | ppm | 100.0 | ppm |
| PL1 | −0.85 | dB | −0.85 | dB |

| | |
|---|---|
| Solvent | CDCl₃ 99.9 atom % D + ~0.1% TMS |

### Preparation 1: Reaction Between ε-Caprolactam and Acrylonitrile in Xylene

123.3 g of ε-caprolactam and 62.5 g of xylene were placed in a 1 1 flask equipped with nitrogen inlet, stirrer, reflux condenser, thermocouple and dropping funnel. Under a light flow of nitrogen, the suspension was heated while stirring at 45-50° C. using an oil bath; once completely solubilised, 0.1841 g of NaOH was added and the temperature was brought to 70° C. Once the sodium hydroxide was solubilised, acrylonitrile (67.4 g) was dripped, taking care to keep the temperature between 7° and 80° C.; the reaction was exothermic. After the addition of the acrylonitrile was completed, it was allowed to react for 2.25 h; a progressive darkening of the solution was noted as the addition reaction progressed.

GC-MS analysis showed a caprolactam conversion of 98.6%, a selectivity of 98.3% and thus a product yield of 96.9%. The basic crude solution was subjected to hydrogenation as described below in Preparation 2. The synthesis was repeated in order to have sufficient product to be subsequently dehydrated.

### Preparation 2: Hydrogenation of Crude Solution in Xylene

In a 250 ml autoclave equipped with a mechanical turbine stirrer, heating mantle, catalyst basket, inlet for gases and liquid streams, 30 g of CTZ1 catalyst was introduced at room temperature into the dedicated catalyst basket and activated in a hydrogen atmosphere.

Activation of the catalyst was carried out by first flushing it with nitrogen at atmospheric pressure, after which the reactor was heated to 150° C. with a temperature ramp of 25-50° C./h, and once this temperature had been reached, hydrogen was supplied at a flow rate of 30 ml/min, thus raising the temperature to 180° C.

At this point, the hydrogen flow rate was increased by progressively reducing the nitrogen flow rate until the gas flushing was completely hydrogen-based (flow rate 200 ml/min). Under these temperature and flow rate conditions, activation continued for 18 hours, after which the nitrogen current was restored (and at the same time the hydrogen current was reduced) in order to maintain the catalyst in an inert atmosphere, gradually cooling the system to room temperature.

To 143.9 g of the solution obtained from Preparation 1, 4.5 g of H₂O was added (approx. 3% of the total) and then loaded into the reactor; the lines were then washed by introducing a further 20.1 g of xylene.

The pressure of the reactor was increased to 21 barA by driving the stirrer motor (750 rpm) and turning on the heater and setting an internal temperature of 130° C.

In the meantime, pressurisation with hydrogen was continued, thus reaching a pressure of 41 barA at the desired temperature. It was hydrogenated at this pressure as long as there was a hydrogen flow of about 0.2-0.3 L/h in line towards the reactor. The volume of hydrogen introduced into the reactor was also indicated by means of the counter and compared with the stoichiometric quantity calculated based on the amount of nitrile introduced. Finally, the product was cooled and discharged.

GC-MS analysis showed a conversion of the nitrile product of 96.1%, a selectivity of 99.3% and thus a yield in the products N-(3-aminopropyl)-ε-caprolactam and DBU (1,8-Diazabicyclo [5.4.0]undec-7-ene) of 95.4%. The synthesis was repeated a two more times in order to have sufficient product to be able to subsequently dehydrate.

### Example 1: Dehydration of Crude Solution in Xylene on Boron Phosphate

The solution from Preparation 2 (138 g) was introduced into a flask (containing a few glass balls), which was connected to a Dean-Stark apparatus equipped with a bubble cooler. One gram boron phosphate was then added, obtained, according to the technique known to those skilled in the art, by evaporation of a solution of boric acid and phosphoric acid (molar ratio 1:1) and calcination at 450° C. for 10 hours. The flask was heated to 170° C.; the water formed by the reaction was separated while the solvent was recovered. After approximately 4 h, the flask was then cooled and the contents were subjected to GC-MS analysis.

From the analysis, the conversion of N-(3-aminopropyl)-ε-caprolactam, selectivity and DBU yield was calculated as shown in Table 1.

### Example 2: Dehydration of Crude Solution in Xylene on Sulfated Zirconia

The example was conducted as in example 1 but using one gram of sulfated zirconium oxide (“Zirconium oxide, catalyst support, sulfated” by Alfa Aesar, cod. 45600, 3 mm pellets) instead of Boron Phosphate.

From the analysis, the conversion of N-(3-aminopropyl)-ε-caprolactam, selectivity and DBU yield was calculated as shown in Table 1.

### Example 3: Dehydration of Crude Solution in Xylene on Cerium Oxide

Example 3 was conducted as example 1 but with one gram of cerium oxide instead of boron phosphate.

Cerium oxide was prepared in the laboratory according to the following process: 500 g of a commercial aqueous solution of approximately 30% ammonium hydroxide (NH₄OH), (28%-30% NH₃ Basis ACS reagent Aldrich) was added to 500 g of water in a first 3-litre beaker equipped with a Teflon crescent-blade stirrer and an electrode was introduced for pH measurement.

The pH meter used is the Metrohom model 780 equipped with Metrohm glass pH electrode model 6.0248.030. In a second 2 litre beaker provided with a magnetic anchor stirrer, a solution was prepared by introducing 100 g of cerium nitrate hexahydrate (Aldrich, code 238538, purity 99%) to 1000 g of water: the cerium nitrate hexahydrate was then dissolved through vigorous stirring at room temperature (25° C.). The solution obtained was inserted into a dropping funnel and fed drop by drop, in 2 hours, to the ammonium hydroxide solution described above, contained in the 3 litre beaker, with constant vigorous stirring. The pH of the suspension obtained was equal to 10.2. The solid in suspension was filtered, washed with 2 litres of water, and then dried in a stove, at 120° C., for 2 hours. The synthesis was repeated until 2000 g of solid were obtained. 1270 g of the solid thus obtained, subject to screening at 0.125 mm, were inserted into an extruder to which 175.9 g of 25% ammonium hydroxide (NH₄OH) solution were also added (obtained by diluting the solution to 28%-30% NH₃ Basis ACS reagent Aldrich) using a Watson Marlow peristaltic pump model 323 DU/D set to 5 rpm. After this, 158 g of demineralized water were also added, thus providing the right consistency for extrusion. The “pellets” obtained at the outlet of the extruder were dried in air and, subsequently, a 100 g portion was calcined at 800° C. with a 1° C./minute ramp to 800° C., followed by isotherm in temperature for 6 hours. The calcined solid was granulated and screened and the fraction of granules of size comprised between 0.5 mm and 1 mm was used as a catalyst.

From the analysis, the conversion of N-(3-aminopropyl)-ε-caprolactam, selectivity and DBU yield was calculated as shown in Table 1.

Table 1 shows the summary data of the previous examples. Finally, it is understood that further modifications and variations to the process as described and illustrated herein may be made which are not specifically mentioned in the text, but which are to be considered as obvious variations of the present disclosure within the scope of the appended claims.

### Calculation of Conversions (Conv), Selectivity (Sel) and Yield (GC-MS)

$$conv \% = \frac{mol reagente iniziale - mol reagente residuo}{mol reagente iniziale} * 100$$

$$sel \% = \frac{mol prodotto ottenute}{mol reagente reagite} * 100 \qquad resa \% = \frac{conv \% * sel \%}{100}$$

$$\begin{matrix}Where : \\ mol = number of moles\end{matrix}$$

TABLE 1
Dehydration reactions

| | Example 1 | Example 2 | Example 3 |
|---|---|---|---|
| mass cat [g] | 1.00 | 1.00 | 1.00 |
| mass solution | 138 | 138 | 138 |
| (NH₂) [g] | | | |
| Catalyst | Boron phosphate | Sulfated zirconium | Cerium oxide |
| | | oxide | |
| T [° C.] | 160-170 | 160-170 | 160-170 |
| P [Barg] | 0 | 0 | 0 |
| conversion (%) | 96.4 | 95.8 | 80.2 |
| selectivity (%) | 98.3 | 99.2 | 98.9 |
| yield (%) | 94.8 | 95.0 | 79.3 |

Catalysts according to the present disclosure have been shown to be advantageous over known homogeneous p-toluenesulfonic acid catalysts in that, being solid, they remain on a fixed bed, whereas known homogeneous catalysts are lost at the end of synthesis. Furthermore, the catalysts according to the present disclosure show very good selectivity towards DBU.

1. A method for preparing amidines or derivatives thereof of formula 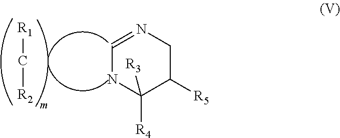
starting from a compound of formula: 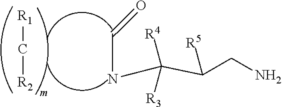
wherein
R1 is H or an aliphatic hydrocarbon group, optionally substituted, having from 1 to 5 carbon atoms, and is more preferably H;
R2 is H or an aliphatic hydrocarbon group, optionally substituted, having from 1 to 5 carbon atoms, and is more preferably H;
R3 is H or an aliphatic hydrocarbon group, optionally substituted, having from 1 to 5 carbon atoms, and is more preferably H;
R4 is H or an aliphatic hydrocarbon group, optionally substituted, having from 1 to 5 carbon atoms, and is more preferably H;
R5 is H or an aliphatic hydrocarbon group, optionally substituted, having from 1 to 5 carbon atoms, and is more preferably H;
m is an integer from 3 to 7,
wherein said process comprises a step of subjecting said compound of formula to dehydration in the presence of an acid catalyst,
wherein the acid catalyst is selected from heterogeneous acid catalysts based on, or comprising, one or more compounds selected from the group consisting of:
cerium oxide (ceria) and/or salts thereof, preferably phosphates;
boron phosphate;
phosphotungstic acid;
zirconium salts, preferably phosphates, and/or modified zirconium oxides, preferably zirconium oxide sulfated.

2. The method according to claim 1, wherein the catalyst is selected from catalysts based on, or comprising, boron phosphate, cerium oxide, zirconium oxide sulfated, zirconium salts or combinations thereof.

3. The method according to claim 1, wherein the catalyst is supported and/or bound on an inert carrier, preferably selected from pumice, graphite and silica.

4. The method according to claim 1, wherein the dehydration reaction is carried out in the absence of a solvent, at a temperature comprised between 9° and 270° C., while continuously removing water produced during dehydration.

5. The method according to claim 1, wherein the dehydration reaction is carried out in the presence of a solvent selected from xylenes and ethylbenzene, at a temperature comprised between 9° and 270° C., while continuously removing water produced during dehydration.

6. The method according to claim 1, wherein the dehydration reaction is carried out continuously in a tubular-type reactor, equipped with a heating system and a condensing system comprising a condenser which condenses most of the produced water and which sends the condensates to a phase separator, and said method comprising separating any traces of organic products in the phase separator and reintroducing the same into the dehydration reactor.

7. The method according to claim 1, wherein the dehydration reaction is carried out continuously in a tubular-type reactor with a WHSV (Weight Hourly Space Velocity, with respect to the entire reagent mixture) comprised between 1 and 50 h⁻¹.

8. The method according to claim 1, wherein the dehydration reaction is carried out at a pressure comprised between 0.08 and 5 BarA.

9. The method according to claim 1, wherein the compound of formula (V) is 1,8-Diazabicyclo [5.4.0]undec-7-ene (DBU).
